(19) United States
(12) Patent Application Publication
(10) Pub. No.: US 2016/0058304 A1
(43) Pub. Date: Mar. 3, 2016

(54) SYSTEM AND METHOD FOR VESSEL ARCHITECTURAL IMAGING

(71) Applicants: Kyree Eeg Emblem, Boston, MA (US); Gregory A. Sorensen, Belmont, MA (US); Rakesh K. Jain, Wellesley, MA (US); Bruce R. Rosen, Lexington, MA (US); Kim Mouridsen, Hjortshoj (DK); Atle Bjornerud, Oslo (NO)

(72) Inventors: Kyree Eeg Emblem, Boston, MA (US); Gregory A. Sorensen, Belmont, MA (US); Rakesh K. Jain, Wellesley, MA (US); Bruce R. Rosen, Lexington, MA (US); Kim Mouridsen, Hjortshoj (DK); Atle Bjornerud, Oslo (NO)

(21) Appl. No.: 14/773,632

(22) PCT Filed: Mar. 10, 2014
(86) PCT No.: PCT/US2014/022331

§ 371 Date: Sep. 8, 2015

Related U.S. Application Data
(60) Provisional application No. 61/775,001, filed on Mar. 8, 2013.

(51) Int. Cl. A61B 5/02; G01R 33/56; A61B 5/00

(52) U.S. Cl. CPC A61B 5/02007; A61B 5/7282; A61B 5/4842; A61B 5/4848; A61B 5/7246; G01R 33/5601; A61B 2576/02

(57) ABSTRACT

A system and method for generating a report regarding a vascular health status of a subject being imaged using a magnetic resonance imaging (MRI) system includes receiving a plurality of MRI datasets, each MRI dataset acquired from the a portion of the subject including a vascular structure. The process also includes analyzing the MRI datasets to identify at least one of a temporal shift and a MR signal variation between the MRI datasets, correlating the at least one of the temporal shift and the MR signal variation to a vascular health status, and generating a report indicating the vascular health status of the subject.

### CROSS-REFERENCE TO RELATED APPLICATIONS

This application is based on, claims priority to, and incorporates herein by reference in its entirety U.S. Provisional Application No. 61/775,001, filed Mar. 8, 2013, and entitled, “SYSTEM AND METHOD FOR VESSEL ARCHITECTURAL IMAGING.”

### STATEMENT REGARDING FEDERALLY SPONSORED RESEARCH

This invention was made with government support under K25AG029415, R21CA117079, R01CA129371, K24CA125440, UL1RR025758, P01CA80124, UL1RR025758, S10RR023401, S10RR019307, S10RR019254, S10RR023043, S10RR021110, R01CA137254, 5R01NS060918, and UL1RR025758 awarded by the National Institutes of Health. The government has certain rights in the invention.

### BACKGROUND OF THE INVENTION

The field of the invention is imaging methods and systems. More particularly, the invention relates to systems and methods for analyzing vascular health of a subject using an imaging system, such as a magnetic resonance imaging (MRI) system.

Anti-angiogenic therapeutic agents target solid tumors by vessel pruning and normalization of vascular structure and function thereby contributing to improved outcome of simultaneously administered chemo-, radiation- and immuno-therapies. In a trial using cediranib, an oral pan-vascular endothelial growth factor (VEGF) receptor kinase inhibitor, patients with recurrent glioblastomas whose tumor perfusion increased during treatment survived approximately 6 months longer compared to those whose perfusion did not increase. Although promising, the exact microvascular mechanism by which these drugs increase perfusion and subsequently improve survival in patients is not fully understood.

Thus, it would be desirable to have a system and method for analyzing microvascular structures and mechanism in vivo.

### SUMMARY OF THE INVENTION

The present invention overcomes the aforementioned drawbacks by providing a system and method for assessing variations in transverse relaxation rates as a function of vascular structure, including microvascular structures, to gain new insight into such structures. That is, the present disclosure provides a system and method for using an magnetic resonance imaging (MRI) system to analyze temporal shift in the MR signal and form the basis for vessel caliber estimation. This technique will be referred to as vessel architectural imaging (VAI). To this end, the present disclosure provides a new biomarker and means to analyze the biomarker (VAI) to aid in various clinical applications and decisions, including gaining insights into the treatment of cancer patients.

In accordance with one aspect of the disclosure, a method for generating a report regarding a vascular health status of a subject being imaged using a magnetic resonance imaging (MRI) system is provided. The method includes receiving a plurality of MRI datasets, each MRI dataset acquired from the a portion of the subject including a vascular structure and analyzing the MRI datasets to identify at least one of a temporal shift and a MR signal variation between the MRI datasets. The method also includes correlating the at least one of the temporal shift and the MR signal variation to a vascular health status and generating a report indicating the vascular health status of the subject.

In accordance with another aspect of the disclosure, a magnetic resonance imaging (MRI) system is disclosed that includes a magnet system configured to generate a polarizing magnetic field about at least a region of interest (ROI) of a subject arranged in the MRI system. The system further includes a plurality of gradient coils configured to apply a gradient field with respect to the polarizing magnetic field and a radio frequency (RF) system configured to apply RF excitation fields to the subject and a acquire MR image data therefrom. The system also includes a computer programmed to control the plurality of gradient coils and the RF system. Accordingly, the computer is programmed to control the plurality of gradient coils and the RF system to perform two different pulse sequences and acquire two different datasets from the subject. The computer is also programmed to compare the datasets to identify at least one of a temporal shift and an MR signal variation between the datasets and correlate the temporal shift to a vascular health status.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an MRI system that employs the present disclosure.

FIG. 2 is a flow chart setting forth some examples of steps that can be performed in accordance with the present disclosure using the system of FIG. 1.

FIG. 3A is an example of a vessel vortex curve in accordance with the present disclosure.

FIGS. 3B and 3C are examples of further vessel vortex curves in accordance with the present disclosure.

FIG. 3D is a graphic illustration of a series of vessel vortex curves in accordance with the present disclosure.

FIG. 3E is a series of vessel vortex curves representing a longitudinal study in accordance with the present disclosure.

### DESCRIPTION OF THE INVENTION

When a substance such as human tissue is subjected to a uniform magnetic field (polarizing field B₀), the individual magnetic moments of the excited nuclei in the tissue attempt to align with this polarizing field, but precess about it in random order at their characteristic Larmor frequency. If the substance, or tissue, is subjected to a magnetic field (excitation field B₁) that is in the x-y plane and that is near the Larmor frequency, the net aligned moment, M_{z}, may be rotated, or “tipped”, into the x-y plane to produce a net transverse magnetic moment Mₜ. A signal is emitted by the excited nuclei or “spins,” after the excitation signal B₁ is terminated, and this signal may be received and processed to form an image.

In MRI systems, the excited spins induce an oscillating sine wave signal in a receiving coil. The frequency of this signal is near the Larmor frequency, and its initial amplitude, A₀, is determined by the magnitude of the transverse magnetic moment Mₜ. The amplitude, A, of the emitted NMR signal decays in an exponential fashion with time, t.

An important factor that contributes to the amplitude A of the NMR signal is referred to as the spin-lattice relaxation process that is characterized by the time constant T₁. It describes the recovery of the net magnetic moment M to its equilibrium value along the axis of magnetic polarization (z-magnetization). The difference in T₁ between tissues can be exploited to provide image contrast.

The T₂ time constant is referred to as the “spin-spin relaxation” constant, or the “transverse relaxation” constant. The T₂ constant is inversely proportional to the exponential rate at which the aligned precession of the spins would dephase after removal of the excitation signal B₁ in a perfectly homogeneous field. The T₁ time constant is longer than T₂ and, in fact, the T₁ time constant is much longer than T₂ in most substances of medical interest.

Two pulse sequences that are used to manipulate these relaxation times to generate useful clinical images are the spin-echo pulse sequence and the gradient-echo pulse sequence. These pulse sequences are versatile and, thus, are used in a wide variety of clinical applications.

MRI is the modality of choice for soft tissue imaging in vivo. In addition to measures of perfusion and blood volume, newer MRI techniques can estimate microvascular vessel caliber, thereby providing further insight into tissue microvascularity. Using these techniques, vessel caliber is estimated by comparing the changes in observed proton relaxation from simultaneously acquired contrast-enhanced, gradient-echo and spin-echo MRI acquisitions. The gradient-echo and spin-echo readouts have different sensitivity to the so-called “susceptibility effect.” The magnetization induced in a medium when exposed to a magnetic field and the highly susceptibility-sensitive gradient-echo images are sensitive to both microscopic and macroscopic vessels, whereas spin-echo images are predominantly sensitive to microscopic vessels (radius <10 μm).

As will be described, the present invention identifies and utilizes a temporal shift in the MR signal that forms the basis for vessel caliber estimation. The techniques that support the systems and methods described below will be referred to as vessel architectural imaging (VAI).

In practice, vessel caliber by MRI is assessed using the quotient of gradient-echo to spin-echo blood volume or direct assessment of the point-by-point difference in the contrast agent-enhanced relaxation rate curves. However, the present disclosure recognizes that, depending on the hemodynamic properties of the tissue, the different sensitivities of the gradient-echo and spin-echo images to the susceptibility effect will result in an apparent variation in the respective MRI signal readouts. That is, the outcome of this is a relative shift in the shapes and peak positions of the two relaxation rate curves.

This can be visualized in a parametric plot and, depending on tissue type, the pair-wise gradient-echo and spin-echo data points may form a vortex curve of a certain shape and transverse in a clockwise or counter-clockwise direction. Prior to the present disclosure, the origin of this phenomenon, its exact relationship to the underlying tissue, and its implication for imaging in cancer patients have not been recognized. However, as will be described VAI was used to demonstrate that an anti-angiogenic therapy led to a reduction in tumor vessel calibers, improved hemodynamic efficiency and oxygen saturation levels, and correlated with prolonged survival.

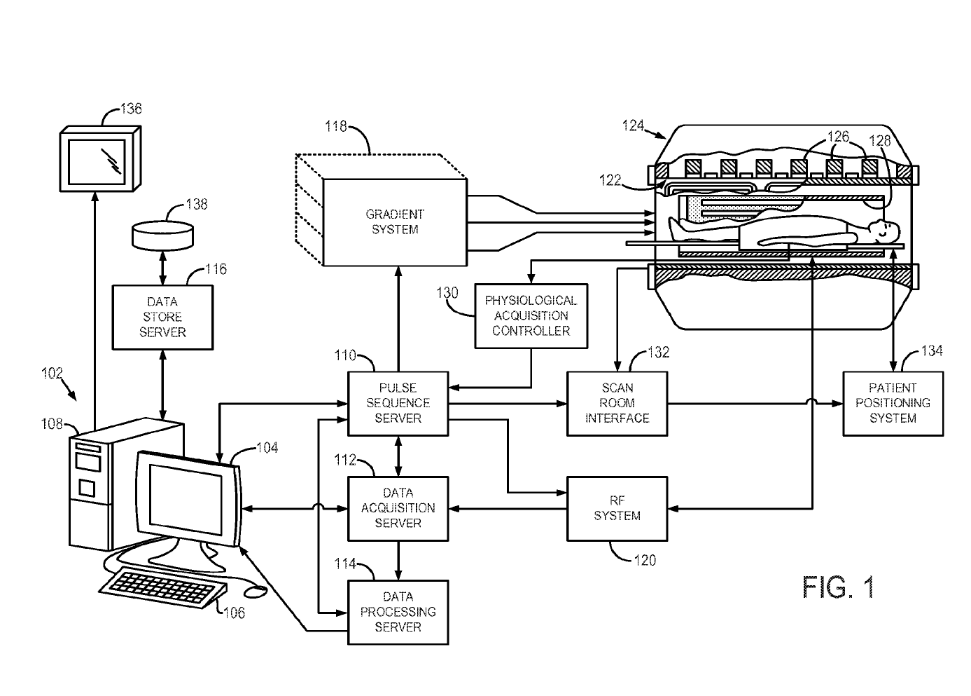

Referring to FIG. 1, an exemplary MRI system 100 for use with the present invention and configured to carry out a process in accordance with the present invention is illustrated. The MRI system 100 includes a workstation 102 having a display 104 and a keyboard 106. The workstation 102 includes a processor 108, such as a commercially available programmable machine running a commercially available operating system. The workstation 102 provides the operator interface that enables scan prescriptions to be entered into the MRI system 100. The workstation 102 is coupled to four servers: a pulse sequence server 110; a data acquisition server 112; a data processing server 114, and a data store server 116. The workstation 102 and each server 110, 112, 114 and 116 are connected to communicate with each other.

The pulse sequence server 110 functions in response to instructions downloaded from the workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 that includes a polarizing magnet 126 and a whole-body RF coil 128.

RF excitation waveforms are applied to the RF coil 128, or a separate local coil (not shown in FIG. 1), by the RF system 120 to perform the prescribed magnetic resonance pulse sequence. Responsive MR signals detected by the RF coil 128, or a separate local coil (not shown in FIG. 1), are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays (not shown in FIG. 1).

The RF system 120 also includes one or more RF receiver channels. Each RF receiver channel includes an RF amplifier that amplifies the MR signal received by the coil 128 to which it is connected, and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} Eqn. (1);

and the phase of the received MR signal may also be determined:

$$ϕ = {tan}^{- 1} ( \frac{Q}{I} ) . \qquad Eqn . ( 2 )$$

The pulse sequence server 110 also optionally receives patient data from a physiological acquisition controller 130. The controller 130 receives signals from a number of different sensors connected to the patient, such as electrocardiograph (“ECG”) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled. The data acquisition server 112 may also be employed to process MR signals used to detect the arrival of contrast agent in a magnetic resonance angiography (“MRA”) scan. In all these examples, the data acquisition server 112 acquires MR data and processes it in real-time to produce information that is used to control the scan.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network to other facilities.

The present disclosure recognizes that an MRI system, such as described with respect to FIG. 1 can be used to assess variations in transverse relaxation rates for the gradient-echo and spin-echo signals as a function of contrast agent concentration and microvascular structure to yield highly-useful clinical indicators and guide clinical decisions.

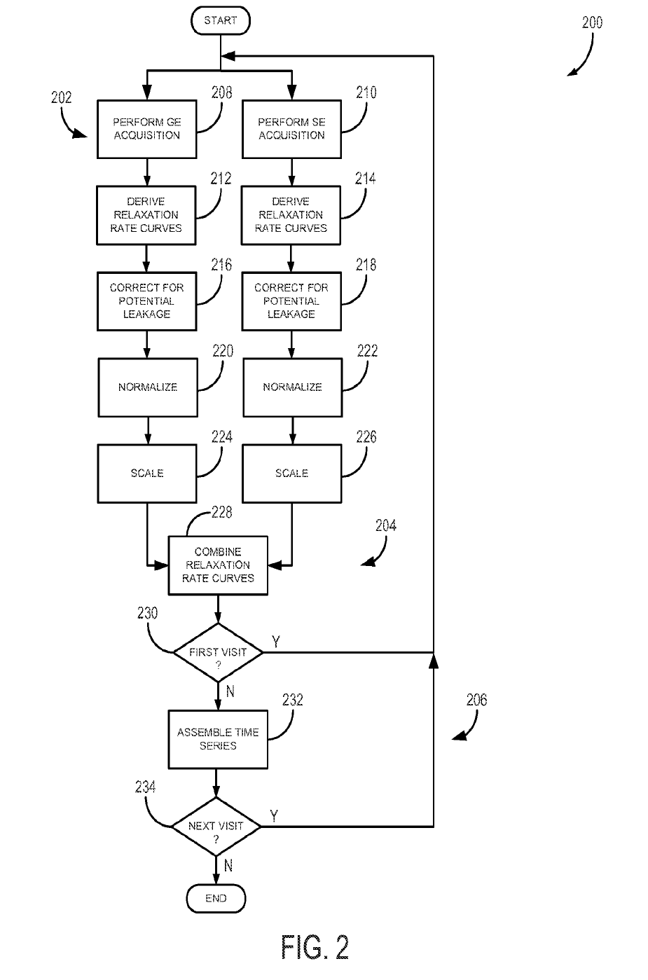

Referring to FIG. 2, a schematic illustration of a process 200 for performing a VAI analysis procedure is illustrated. Generally, the figure can be divided into three distinct sub-concepts, generally, designated as a first sub-process 202, a second sub-process 204, and a third sub-process 206.

First, as generally designated at the first sub-process 202, a gradient-echo (GE) acquisition is performed at process block 208 and spin-echo (SE) acquisition is performed at process block 210. At process blocks 212 and 214, respectively, relaxation rate curves are derived from the GE and SE images using kinetic models. That is, parametric vessel vortex curves can be derived by point-by-point parametric plots of the GE and SE relaxation rate curve. Each set of respective relaxation rate curves may be corrected for potential contrast agent extravasation (process blocks 216, 218), normalized by gamma-variate fitting (process blocks 220, 222), and scaled to reference tissue to correct for global systemic effects (process blocks 224, 226).

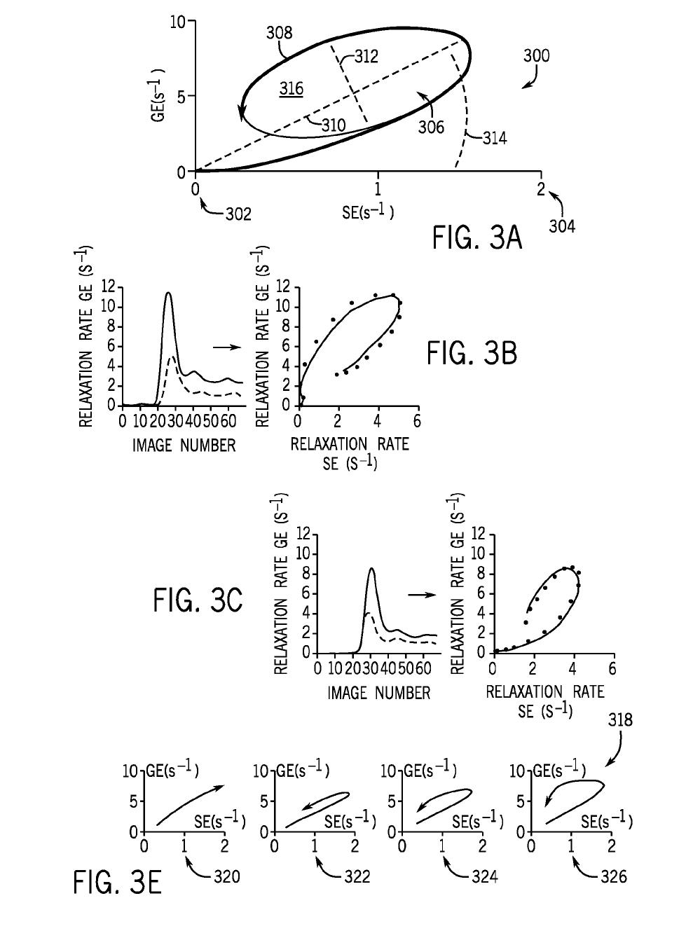

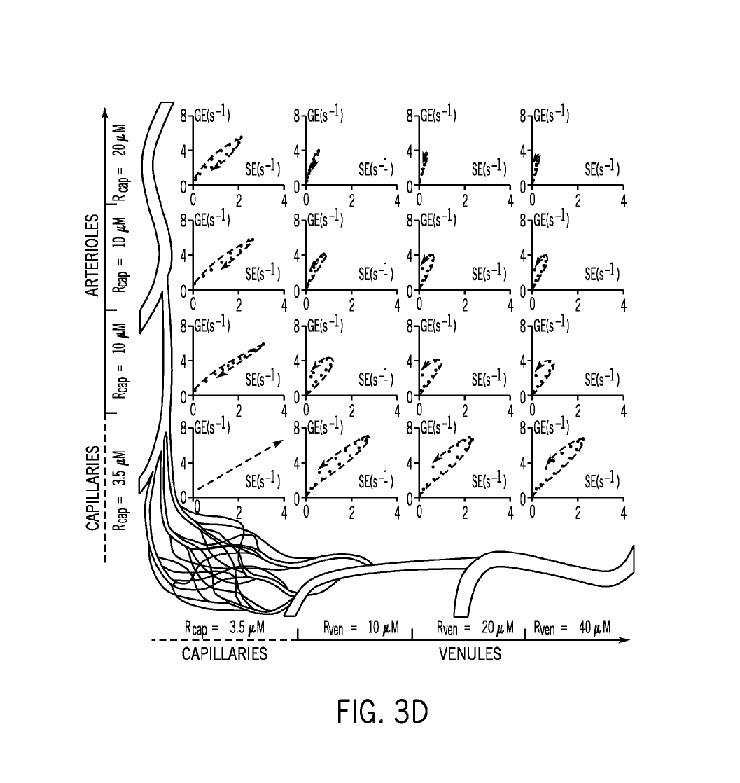

At the second sub-process 204, the GE and SE curves are combined at process block 228, for example, using a scatter plot referred to as a “vessel vortex curve.” An example of a vessel vortex curve 300 is illustrated in FIG. 3A. As illustrated, The vessel vortex curve is created by scatter plotting the SE and GE data with GE on one axis 302 and SE on the other axis 304. Examining the resulting plot 306, a “vessel vortex direction” 308 can be identified by determining the direction the point-by-point scatter plot propagates (counter-clockwise in FIG. 3A). A long axis 310 of the vessel vortex curve 308 can be found by a linear fit (using least squares estimation or similar). Also, a short axis 312 of the vessel vortex 306 is the maximum length of a straight line perpendicular to the long axis 310. An increase in the long axis 310 is equivalent to an increase in an area under the relaxation rate curve, which is the traditional measure of volume fraction Vf (˜blood volume). A slope value 314 is the gradient of the long axis 310 and describes its steepness. An area 316 of the vessel vortex 306 is the best fit of the curve area. However, a corrected vessel vortex area can be the estimated as the area 316 divided by the length of the long axis 310. This correction will account for systemic or local, tissue-specific variations in Vf. A vessel vortex curve can be created for all relevant image voxels.

That is, referring to FIGS. 3B and 3C, the present disclosure recognizes that different sensitivities of the gradient-echo and spin-echo images to the susceptibility effect will result in an apparent variation in the respective MRI signal readouts. This difference can be used in the above described process to translate a given set of GE and SE data into vessel vortex curves. As illustrated in FIG. 3B, in areas with fast inflow of the contrast agent, such as in the feeding branches of the middle cerebral artery, the GE signal peaks earlier than the SE signal resulting in a clockwise vortex when plotting the relaxation rate curves in a point-by-point parametric plot. Correspondingly, as illustrated in FIG. 3C, in slow inflow areas, for example in the venules leading to the internal cerebral veins, the SE signal peaks earlier than the GE signal resulting in a counter-clockwise vortex. The contrast agent-induced relaxation rates in FIGS. 3B and 3C are scaled relative to their baseline rates (prior to contrast agent arrival), and will increase and decrease with a full-width, half maximum proportional to the mean transit time. Volume fraction (Vf) is defined as the area under the relaxation rate curves (percentage of blood in the image voxel˜blood volume), whereas perfusion (˜flow) can be estimated using the central volume principle stating that Vf is the product of flow and mean transit time.

Thus, referring to FIG. 3D, parametric vessel vortex curves are shown for different vessel combinations. For all vessel combinations illustrated, Vf was kept at 3.5 percent, where an increase in vessel caliber (distension) implies a subsequent reduction in vessel density (negative recruitment). The SO2 levels were kept at normal values (arterioles at 90-95 percent %, capillaries and venules at 50 percent). As illustrated, the vessel vortexes transverse in a counter-clockwise direction if, the vascular system contains both slow inflow, larger-caliber venule-like vessel components and faster inflow, smaller-caliber arteriole-like or capillary-like vessel components. In contradistinction, if the vascular system consists of arterioles and capillaries only—or, for some geometrical, pathological or physiological reason, fast inflow arterioles with larger calibers than venules—the vessel vortexes transverse in a clockwise direction. For vessels of identical calibers, because of differences in tissue-specific oxygen saturation (SO2) levels, the vessel vortexes transverse in a counter-clockwise direction if both arterioles and venules are included. However, if all vessels have identical calibers and SO2 levels, there is no vortex. Similarly, if a vascular system with a fixed SO2 level contains arterioles, capillaries or venular structures only, there is no vortex—even if the vessels have different radii.

The shape of the vessel vortex curve depends not only on the vessel types included, but also on their relative difference in vessel radius. As discussed above with respect to FIG. 3A The slope of the vortex curve is assumed proportional to the vessel caliber, and tilted towards the gradient-echo axis for vascular systems with larger average vessel calibers.

Referring again to FIG. 2, the above-described process can be performed for a given visit and, thus, provide vessel voxel curves for that study. Also, at decision block 230, the third sub-process 206 can be employed to perform the above-described process across a series of visits. That is, the first sub-process 202 and the second sub-process 204 can be repeated upon each visit of an given subject to create a series of relaxation rate curves at process block 232.

For example, FIG. 3E provides an example of a series of vessel voxel curves 318 acquired from data sets associated with a first visit 320, a second visit 322, a third visit 324, and a fourth visit 326. This series of vessel voxel curves 318 may correspond to particular voxels across each study or may the longitudinal analysis may be of average values across a region of interest (ROI) or, for example, a tumor.

Thus, referring again to FIG. 2, the process may be repeated at decision block 234 across a series of sequential visits and corresponding imaging studies to perform an analysis over time.

### EXAMPLES

The intravascular fraction of tissue can be approximated by randomly oriented water impermeable cylinders with a defined blood volume fraction (Vf), radius (R), water diffusion (D) and susceptibility difference (Δ_{X}) between the intra-cylindrical and extra-cylindrical space. In one study, vessels were modeled as infinite cylinders under the assumption that the average proton diffusion length during the observation time, equal to the echo-time (TE) of the respective GE and SE sequences, is much shorter than the typical vessel segment length. The z-component of the magnetic field perturbation of the external magnetic field B0 (oriented along the horizontal z-axis of the MR magnet) due to each segment can then be approximated by:

$$\frac{Δ {B}_{Z} ( ϕ , θ}{{B}_{0}} \{ \begin{matrix}2 πΔ {X ( \frac{R}{r} )}^{2} cos 2 {ϕsin}^{2} θ , & r \geq R \\ \frac{2 π}{3} Δ X ( 3 {cos}^{2} θ - 1 ) , & r < R ;\end{matrix} \qquad Eqn . ( 3 )$$

where θ is the angle between B0 and the cylinder axis and (φ, r) are the polar coordinates of the proton location relative to the projection of B0 in a plane orthogonal to the cylinder axis.

A single proton was placed at the origin of the closed simulation space and allowed to randomly diffuse in a plane orthogonal to B0 through the intra- or extra-cylindrical space with total diffusion duration equal to the echo-time. The random walk was simulated by arbitrarily changing the orientation of the spin every 0.1 ms using a Gaussian displacement distribution (with mean; μ=0 and variance; σ=√2DΔt) along the orthogonal directions at each time step. The magnetic field perturbation at the proton position from a predefined set of cylinders, and the corresponding phase shift, were recorded every 0.5 ms. This procedure was repeated for n=5000 protons and the complex signal due to the accumulated phase of all protons were defined as:

$$S ( t ) = \frac{1}{N} {\sum }_{n = 1}^{N} {e}^{{iφ}_{n} ( t )} ; \qquad Eqn . ( 4 )$$

where (t) n φ is the phase of the n-th proton at time t. The proton phase accumulated during a time step from the presence of each cylinder was given by Δφ n(t)=γΔB zΔt, where γ is the gyromagnetic ratio.

For estimations of Δ_{X} as a function of contrast agent concentration, the baseline magnetic susceptibility of fully oxygenated blood and tissue was assumed to be equal and Δ_{X} directly proportional to [C]_{X}Gd, where [C] is the intravascular gadolinium concentration and _{X}Gd=0.32*10−6 mM−1 as previously shown. It is noted that intra-vascular, iron-oxide contrast agents, as an example of non-gadolinium-based contrast agents, may likewise be used. The transverse relaxation effect due to deoxygenated blood was modeled by inclusion of an additional intra-and extra-vascular susceptibility difference in the resulting arterial, capillary and venous relaxation rate curves which could be varied between Δ_{X}dHb=2.5*10−6 for fully deoxygenated blood and ΔₓdHb=0 for fully oxygenated blood. The relationship between arteriole and venule brain hematocrit were considered linear for our data and thus negligible. The corresponding change in relaxation rate (shown for GE) was estimated by assuming a mono-exponential signal decay as a function of diffusion time:

ΔR*₂=−ln(|S(TE)|)/TE Eqn. (5);

where ∥ is the modulus of the signal. For estimation of SE relaxation rate changes, the phase dispersion was inverted at t=TE/2 to account for the refocusing effect of the 180 degree radio frequency pulse. The SE signal decay was then similarly measured at time TE and converted to the corresponding change in relaxation rate according to equation (5). The effect of red blood cell velocity combined was not included in our simulations. Unlike normal tissue, blood velocities in tumor do not strictly depend on vessel calibers. However, previous simulations suggest that the effect of red blood cell velocity is expected to be equal for the gradient-echo and spin-echo relaxation rate curves with minimal variance for changes in velocity about or above physiologic capillary velocities (≧0.2 cm s−1). Furthermore, it should be noted that the random walk model is limited in that it only simulates relaxation effects due to proton diffusion orthogonal to the external magnetic field and does not model the full effect of three-dimensional displacements. However, given the random orientation of capillaries, the induced relaxation effect can be assumed to be independent of z-position (position along B0) and the two-dimensional model has previously been shown to provide relaxation rate estimates in very good agreement with in vivo data obtained with an intravascular contrast agent in a rat model.

Representative relaxation rate curves following a simulated contrast agent injection were estimated by coupling the resulting GE and SE relaxation rates at physiologically meaningful values of Vf, radius and water diffusion to synthetic and typical arterial, capillary and venous shaped curves using JSim (National Simulation Resource Physiome initiative). Parametric vessel vortex curves were derived by point-by-point parametric plots of the gradient-echo and spin-echo relaxation rate curves. The effect of contrast agent extravasation due to disrupted blood-brain-barrier was assumed negligible or corrections were made.

The study was approved by the institutional review board and informed consent was obtained from all subjects. Subject data included 13 females and 17 males diagnosed with a recurrent glioblastoma, average age 52 years, range 20-78 years. After study termination, nine subjects received one subsequent cycle of salvage chemotherapy, eight subjects received two cycles, one subject received three cycles, two subjects had undisclosed information on salvage chemotherapy and one subject received stereotactic radiosurgery.

Baseline MRI examinations were acquired prior to therapy onset (days −5 and −1), and then repeated on days 1, 28, 56 and 112 after cediranib (AstraZeneca Pharmaceuticals) anti-angiogenic therapy onset or until disease progression according to the Macdonald criteria. All imaging were performed on a 3 Tesla Magnetom Trio MRI system (Siemens Medical Solutions) as follows:

1. T1-weighted images. Axial images acquired prior to, and after, contrast agent injection (gadopentetate-dimeglumine, Gd-DTPA, Magnevist, Bayer Schering Pharma AG). Repetition-time 600 ms, echo-time 12 ms, slice-thickness 5 mm, inter-slice distance 1 mm, in-plane resolution 0.45:0.45 mm, matrix size 384:512 and 23 slices.

2. T2-weighted (FLAIR) images. Axial images with repetition-time 10 s, echo-time 70 ms, slice-thickness 5 mm, inter-slice distance 1 mm, in-plane resolution 0.60:0.45 mm, matrix size 384:512 and 23 slices.

3. Dynamic contrast enhanced (DCE) images. Axial, fast gradient-echo images with repetition-time 5.7 ms, echo-time 2.73 ms, slice-thickness 2.1 mm, inter-slice distance 0.4 mm, in-plane resolution 2.90:2.00 mm, matrix size 128:87 and 20 slices. After approximately 52 s of imaging, a 0.1 mmol kg−1 dose of Gd-DTPA was injected at 5 cc s−1. Also, spoiled gradient recalled-echo images with five different flip angles (2, 5, 10, 15 and 30 degrees) were initially acquired for T1-mapping.

4. Dynamic susceptibility contrast (DSC) perfusion images. Axial dual-echo echo-planar images with repetition-time 1.33 s, echo-times 34 ms and 103 ms, slice-thickness 5 mm, inter-slice distance 2.5 mm, in-plane resolution 1.70:1.70 mm, matrix size 128:128, 10 slices and 120 volumes. After approximately 85 s of imaging, a 0.2 mmol kg−1 dose of Gd-DTPA was injected at 5 cc s−1.

An experienced neuroradiologist identified tumor by outlining enhancing regions on the contrast-enhanced T1-weighted images and peritumoral vasogenic edema on the FLAIR images. The anatomical MR images were realigned to the DSC and DCE images using normalized mutual information coregistration. On the T1-weighted tumor outlines, areas corresponding to the tumor center and edge were derived using three-dimensional connectivity morphologic analysis in Matlab where an image voxel was assumed to be a center voxel if all neighboring cubical voxels were also outlined as tumor.

The DCE data were processed using custom-made software in Matlab, applying standard approaches to create Ktrans maps, a measure of the permeability that roughly corresponds to wash-in rates of the contrast agent in tissue.

We obtained relaxation rate curves for VAI analysis, perfusion values, blood volumes and mean transit times using established tracer kinetic models on the DSC images, corrected for contrast agent leakage (from blood-brain-barrier breakdown or resection) and fitted to a gammavariate curve for better visualization of vessel vortex effects. It has been speculated that contrast agent leakage is the reason for the clockwise vortex effect. Not correcting for leakage resulted in an average 5 percent drop in the clockwise to counter-clockwise vortex direction ratio with minimal influence on our results. Here, the pre-dose of Gd-DTPA during DCE imaging saturated potential leaky tumor tissue in the DSC images thereby minimizing the influence of leakage-induced T1-shortening effects. Relaxation rate curves not suited for analysis, conveying highly fluctuating time-courses from partial volume effects, voxel shifts and physiological pulsations were excluded from further analysis. Across all 30 subjects, an average of 78.21 percent±12.78 percent (standard deviation) of all tumor voxels met the inclusion criteria. To account for potential global systemic effects from hypertension, tumor relaxation rate curves were scaled with corresponding slice-specific mean, normal-tissue reference curves. In all figures showing vessel vortex curves, the tails of the vortex curves have been cut short to better visualize vortex direction. VAI analysis was performed using custom-made software in Matlab and traditional MRI was analyzed in nordicICE (NordicNeuroLab AS).

A subject was assumed to have an increase (decrease) in voxels with a clockwise vortex direction if the clockwise- to counter-clockwise ratio was higher (lower) than the 95 percent confidence interval of the population arithmetic mean for two consecutive imaging time points. Subjects who did not meet this criterion were treated as having no change in vortex direction ratio. Differences in VAI parameters during therapy were assessed using pairwise Wilcoxons Signed Rank test. Differences in tumor volumes, vessel caliber, permeability, perfusion and mean transit times were assessed using Mann-Whitney tests. Associations between changes in vessel vortex direction ratios and progression-free survival and overall survival were assessed using multinomial logistic regression, Kaplan-Meier survival analysis and Cox regression after adjustments for age, extent of resection, neurological performance, salvage chemotherapy and stereotactic radiosurgery after study termination as well as changes in permeability (Ktrans), T1-weighted contrast-enhanced tumor volume and T2-weighted FLAIR tumor volume prior to, and during, anti-angiogenic therapy. For all tests, P=0.05 was considered significant (with Holm-Bonferroni correction for multiple comparisons) and pixel values below a 5 percent percentile and above a 95 percent percentile were removed prior to analysis to reduce the influence of outliers. Reproducibility tests were assessed using Spearman Rank correlations and Bland-Altman plots. Statistical analysis was performed using SPSS 17 (SPSS Inc.).

As described above, VAI techniques can be used to investigate different blood volume fractions (Vf) and varying levels of SO2. In particular, increased Vf by vessel recruitment results in a proportional increase in the length of the long axis of the vessel vortex curve, an exponential decrease in slope value, and a proportional increase in the corrected vessel vortex area. For vessel distention similar, although less pronounced effects can be observed. The only exception is an exponential increase in slope value with increased Vf for tissue without functioning or missing capillary vessels or for vessel shunting. The vessel vortex direction was not affected by the induced changes in vessel recruitment nor distention.

The resulting vessel vortex curves for varying levels of SO2 can be assessed using different combinations of arterioles, capillaries, and venules. Specifically, the length of the long axis and the slope of the vessel vortex curve increase with increasing levels of deoxygenated blood. In a vascular system with relatively unchanged or fixed vessel calibers and inflow rates, the corrected vessel vortex area reflects the different baseline susceptibility states in oxygenated and deoxygenated blood and thus SO2 levels. Here, under normal conditions (venule calibers >arteriole calibers), the corrected vessel vortex area shows a Gaussian, bell-shaped response to changes in SO2 levels expressed by an increase in the corrected vessel vortex area for increased absolute differences in SO2 levels between well-saturated, oxygenated arterioles (SO2>90 percent) and deoxygenated capillaries and venules (SO2<90 percent). Correspondingly, for anoxic SO2 levels and towards a theoretical and fully deoxygenated hemodynamic environment (arterioles; SO2<75 percent, capillaries and venules; SO2=0 percent), the corrected vessel vortex area decreases.

Such information can be used to improve a variety of clinical applications. For example, one clinical application of VAI was demonstrated by retrospective analysis of 30 human subjects with recurrent glioblastomas enrolled in a Phase II clinical trial of cediranib. Collectively, a significant increase (pair-wise Wilcoxons Signed Ranks test; P<0.05) in the relative number of image voxels with a clockwise vortex direction in the tumor after therapy onset was observed thereby mimicking normal-appearing tissue values.

A more dominant effect was seen in the tumor center (pair-wise Wilcoxons Signed Rank test; P<0.01) compared to the tumor edge. Ten subjects were identified as responders to the anti-angiogenic therapy by a relative increase in image voxels with a clockwise vortex direction compared to the arithmetic mean of all subjects, and at a minimum of two consecutive imaging time points. Twelve subjects were identified as non-responders by a relative decrease in image voxels with a clockwise vortex direction.

Median overall survival for responding subjects was 341 d compared to 146 d for nonresponders. Using Cox regression with time dependent covariates, the relative increase in clockwise vessel vortexes during anti-angiogenic therapy was an independent predictor of progression-free survival and overall survival (P<0.01) and also reflected in significant reductions in the contrast enhanced and FLAIR tumor volumes at day 28 (Mann-Whitney tests; P<0.05). In addition to Vf, no differences in vessel calibers, permeability, SE and GE perfusion (flow) or SE and GE mean transit times were observed between the two groups. For responding subjects and compared to pre-treatment, significant reductions in whole-tumor vessel calibers (pair-wise Wilcoxons Signed Rank test; P<0.01) and subsequent reductions in Vf and corrected vessel loop area in the tumor center was observed (pair-wise Wilcoxons Signed Rank tests; P<0.01). Reproducibility analysis showed minimal variability.

Thus, assessment of the topological and structural heterogeneity of tumor microcirculation is important for monitoring of disease progression and treatment response. Tumor vessels are characterized by increases leakiness and regional, inefficient closed or blind vascular pathways with or without hypoxia. The VAI technique described herein is capable of measuring these effects in vivo, ranging from well-functioning, well-oxygenated normal-appearing tissue to the vascular collapse observed in anoxic tumor tissue.

Study results provide several insights. Overall, the temporal shift in the MR signal can be readily observed with a standard combined GE and SE contrast enhanced MRI acquisition technique. In normal tissue, the resulting vessel vortex curve propagates in a counter-clockwise direction if large, slow inflow vessels and faster inflow vessels with smaller calibers are present. The slope of the vessel vortex curve is indeed influenced by the average vessel caliber of the tissue, but the traditional view of increasing slope values for bigger vessel calibers is dependent on changes in Vf and SO2 levels. The highest slope values were observed for theoretical vessel systems with local shunting, where big disorganized, fast-flow arterioles aberrantly connect to disorganized venous structures. Contrary to vascular systems with functioning capillaries, local shunting will also depict a relatively constant corrected vessel vortex area indicative of little or no difference in oxygen saturation between the tissue types.

The subnormal vascular function and non-uniform branching hierarchy of recurrent glioblastomas was identified by a higher relative ratio of larger-caliber, deoxygenated venule-like vessel compared to other vessel types. This was more pronounced towards the tumor center and in line with a vascular gradient moving from a neoangiogenic tumor border of normal- or dilated vessels towards a hypoxic or anoxic core with scarce, inefficient and very large vessels. During anti-angiogenic therapy, a higher ratio of image voxels with a clockwise vortex direction was observed in responding subjects, mimicking the ratio seen in normal-appearing tissue. This change in vessel vortex direction requires a high quantity of vessels with fast inflow rates combined with a reduction of large vessels with slow inflow. This is consistent with data from studies in animal and human solid tumors where proper doses of anti-angiogenic drugs lead to improved tumor microenvironment and more effective delivery of exogenously administered therapeutics by reduced tumor hyperpermeability and vessel calibers, hypoxia and interstitial fluid pressure and increased vascular pericyte coverage. Correspondingly, the improved microcirculation identified by the VAI technique was predictive of progression-free survival and overall survival. Interestingly, although perfusion plays a key role in the response to therapy, average perfusion values alone could not explain the observed difference between responders and non-responders. This is in line with previous work showing that changes in perfusion are not likely to have a substantial influence on the relaxation rate curves (i.e. vessel vortex curves) and indicates that VAI is a different and potentially more sensitive biomarker than traditional MRI. This is, in part, explained by the VAI's apparent sensitivity to changes in SO2 levels. For responding subjects at day one of treatment, the average vessel vortex curve slope did not increase even though the average Vf decreased—which at a hypothetical fixed SO2 level should have resulted in an increased slope value. This suggests that anti-angiogenic therapy improves and normalizes oxygen concentrations thereby providing benefit to these subjects.

In summary, while traditional MRI of cancer in vivo is confounded by haphazard and heterogeneous vessel architecture with limited or redundant perfusion, the VAI technique exploits these properties and provides further insights into the complex nature of tumor vascularity.

The microvasculature of tumors is abnormal and tortuous with arterio-venous shunts. Shunts are short high-flow vascular pathways that cause parts of the blood flow to bypass capillary regions downstream as well as other, longer pathways. Functional shunts with limited surface area impair delivery of oxygen to the tissue and increase resistance to therapy. VAI can identify arterio-venous shunts in patients with recurrent glioblastomas and help reveal mechanisms of response to anti-angiogenic therapy.

First, to analyze the VAI response to arterio-venous shunts, Monte Carlo simulations of intravascular magnetic susceptibility perturbations were used. A realistic branching of vessels was obtained by using a vessel tree model, where the vessel generations are self-similar and follow Murray's law. Capillaries were gradually removed from the vessel tree and the arterial and venous oxygenation saturation levels (SO2) varied from hyperoxic to anoxic scenarios to mimic impaired oxygen delivery. A parameter coined as the “shunt index” was introduced, defined as the slope of the vessel vortex curve divided by the length of the vortex curve. The slope and length of the vortex curve yields distinct differences in simulations of normal tissue compared to shunting vessels.

Also, MRI data of patients with recurrent glioblastomas enrolled in a Phase II clinical trial of the anti-angiogenic drug cediranib (clinicaltrials.gov, NCT00305656) was evaluated. Gadolinium-based GE and SE dynamic susceptibility contrast MRI was performed at 3T (Siemens) prior to therapy onset (days −5 and −1) and repeated at days 1, 28, 56 and 112 as previously reported. Shunt indexes of patients identified as responders to cediranib by increased perfusion were compared to non-responders with stable or reduced perfusion using Kruskal-Wallis tests and adjusted for variations in blood volume.

Compared to normal tissue, the slope of the vortex curves increase whereas the vortex lengths decrease in shunting tissue. The resulting shunt index for normal tissue is reduced with increasing volume fractions (by distension), whereas the shunt index is stable or increasing in shunts. Pre- and post-therapy shunt index maps overlaid on contrast-enhanced MRIs yield readily distinguishable and reproducible results. Patients identified as responders by vascular normalization had reduced shunt index values at days 1 and 28 (P<0.05) compared to non-responders. Responding patients lived approximately 6 months longer than non-responding patients (median overall survival 348 days vs 169 days; P<0.001).

Using VAI, simulations show higher shunt indexes in shunting tissue compared to normal tissue. Abnormal shunt indexes were also observed in patients with recurrent glioblastomas before anti-angiogenic therapy. Patients with vascular normalization from increase in perfusion had reduced vessel shunting in the tumor during the first month of anti-angiogenic therapy by cediranib. The findings support the hypothesis that restoring mechanisms that counteract shunting underlies the successful normalization of tumor vasculature by anti-angiogenic therapy. Thus, VAI can be used to identify arterio-venous shunts and help reveal mechanism of normalization during antiangiogenic therapy.

The present invention has been described in terms of one or more preferred embodiments, and it should be appreciated that many equivalents, alternatives, variations, and modifications, aside from those expressly stated, are possible and within the scope of the invention.

1. A method for generating a report regarding a vascular health status of a subject being imaged using a magnetic resonance imaging (MRI) system, the method comprising:
receiving a plurality of MRI datasets, each MRI dataset acquired from the a portion of the subject including a vascular structure;
analyzing the MRI datasets to identify at least one of a temporal shift and a MR signal variation between the MRI datasets;
correlating the at least one of the temporal shift and the MR signal variation to a vascular health status; and
generating a report indicating the vascular health status of the subject.

2. The method of claim 1 wherein the plurality of MRI datasets were acquired using at least two different pulse sequences.

3. The method of claim 1 wherein the pulse sequences include at least a spin-echo pulse sequence and a gradient-echo pulse sequence.

4. The method of claim 1 further comprising reconstructing respective images of the subject form each of the MRI datasets and creating respective first-pass curves in response to a contrast agent injected into the subject.

5. The method of claim 4 wherein analyzing the MRI datasets to identify the at least one of the temporal shift and MR signal variation includes comparing the images of the respective images of the subject.

6. The method of claim 5 wherein the comparison is a voxel-by-voxel comparison.

7. The method of claim 1 wherein correlating the at least one of the temporal shift and the MRI signal variation to a vascular heath status includes determining a directional component of the temporal shift.

8. The method of claim 7 wherein correlating the temporal shift includes plotting the temporal shift in a scatter plot with a signal intensity temporal curve of a first of the MR datasets on a first axis of the plot and a signal intensity curve on a second of the MR datasets on a second axis of the plot.

9. The method of claim 7 wherein generating the report includes correlating a first directional component of the temporal shift to a normal vascular health status and correlating a second directional component related to the temporal shift to an abnormal vascular health status.

10. The method of claim 9 wherein the first directional component include a counter-clockwise directional component and the second directional component includes a clockwise directional component.

11. The method of claim 9 wherein the abnormal vascular health status includes at least one of non-normal vascular function and non-uniform branching hierarchy of cancer.

12. The method of claim 7 wherein the abnormal vascular health status is further correlated to a cancer indication.

13. A magnetic resonance imaging (MRI) system comprising:
a magnet system configured to generate a polarizing magnetic field about at least a portion of a subject arranged in the MRI system;
a plurality of gradient coils configured to apply a gradient field to the polarizing magnetic field along each of at least three directions;
a radio frequency (RF) system configured to apply an excitation field to the subject and acquire MR image data therefrom;
a computer system programmed to:
control the plurality of gradient coils and the RF system to perform two different pulse sequences and acquire two different datasets from the subject;
compare the datasets to identify at least one of a temporal shift and an MR signal variation between the datasets; and
correlate the temporal shift to a vascular health status.

14. The system of claim 13 wherein a first of the two different pulse sequences includes a spin-echo pulse sequence.

15. The system of claim 13 wherein a second of the two different pulse sequences includes a gradient-echo pulse sequence.

16. The system of claim 13 wherein the computer system is further programmed to reconstruct respective images of the subject from each of the two different datasets.

17. The system of claim 16 wherein the computer system is further programmed to compare the respective images to identify the temporal shift.

18. The system of claim 17 wherein the computer system is further programmed to perform a voxel-by-voxel comparison to compare the respective images.

19. The system of claim 13 wherein the computer system is further programmed to determine a directional component related to the temporal shift.

20. The system of claim 19 wherein the computer system is further programmed to correlate a first directional component of the temporal shift to a normal vascular health status and correlate a second directional component of the temporal shift to an abnormal vascular health status.

21. The system of claim 20 wherein the first directional component includes a counter-clockwise directional component and the second directional component includes a clockwise directional component.

22. The system of claim 21 wherein the clockwise directional component suggests dominance of arterial vessels and the counter-clockwise directional component suggests dominance of venous vessels.

23. The system of claim 21 wherein a change in average directional component suggests at least one of progression of a disease, response to a therapy, and a vascular normalization.

24. The system of claim 20 wherein the abnormal vascular health status includes at least one of subnormal vascular function and non-uniform branching hierarchy of cancer.

25. The system of claim 13 wherein, to compare the datasets to identify a temporal shift between the datasets, the computer system is further programmed to crate a scatter plot.

26. The system of claim 25 wherein the computer system is further programmed to calculate a corrected area of a vortex of the scatter plot and correlate the area of the vortex to reflect susceptibility states in oxygenated and deoxygenated blood and to oxygen saturation levels of tissue of the subject.

27. The system of claim 26 wherein the computer system is further programmed to identify a change in an average area of the scatter plot vortex as indicative of at least one of disease progression, a response to therapy, and a vascular normalization in the subject.

28. The system of claim 26 wherein the computer system is further programmed to determine a shut index defined as a slope of the vortex divided by the length of the vortex.
